(19) United States
(12) Patent Application Publication
(10) Pub. No.: US 2025/0352607 A1
(43) Pub. Date: Nov. 20, 2025

(54) GLATIRAMER DEPOT SYSTEMS FOR TREATING PROGRESSIVE FORMS OF MULTIPLE SCLEROSIS

(71) Applicant: MAPI PHARMA LTD., Ness Ziona (IL)

(72) Inventors: Uri DANON, Tel Aviv (IL); Nadav BLEICH KIMELMAN, Tel Aviv (IL); Laura POPPER, Tel Aviv (IL); Ehud MAROM, Tel Aviv (IL)

(73) Assignee: MAPI PHARMA LTD., Ness Ziona (IL)

(21) Appl. No.: 19/225,240

(22) Filed: Jun. 2, 2025

Related U.S. Application Data
(63) Continuation of application No. 17/495,458, filed on Oct. 6, 2021, now Pat. No. 12,343,371.
Application No. 17/495,458 is a continuation of application No. 16/497,120, filed on Sep. 24, 2019, now Pat. No. 11,167,003.
(60) Provisional application No. 62/476,794, filed on Mar. 26, 2017.

(51) Int. Cl. A61K 38/02; A61K 9/00; A61K 38/10; A61K 38/16; A61K 39/00; A61K 47/34; A61P 25/00; A61P 25/28; C07K 14/47

(52) U.S. Cl. CPC A61K 38/02; A61K 9/0024; A61K 47/34; A61P 25/00; A61P 25/28; A61K 38/10; A61K 38/16; A61K 39/0008; C07K 14/4713; G01N 2800/285

(57) ABSTRACT

The present invention provides methods for treating or ameliorating primary progressive multiple sclerosis (PPMS) or secondary progressive multiple sclerosis (SPMS) and related symptoms by administering or implanting a depot formulation comprising glatiramer salts, such as glatiramer acetate (GA).

### CROSS-REFERENCE TO RELATED APPLICATIONS

The present application is a Continuation of U.S. patent application Ser. No. 17/495,458 filed on Oct. 6, 2021, which is a Continuation of U.S. patent application Ser. No. 16/497,120 filed on Sep. 24, 2019, and issued as U.S. Pat. No. 11,167,003, which is a national stage filing under 35 U.S.C. § 371 of PCT/IL2018/050340, filed on Mar. 25, 2018, and claims the benefit of priority to U.S. Provisional Application No. 62/476,794, filed on Mar. 26, 2017. Each application is incorporated herein by references in their entirety.

### FIELD OF THE INVENTION

The present invention relates to depot formulations and other implantable systems for prolonged release of glatiramer acetate or other pharmaceutically acceptable salts of glatiramer for treating primary progressive multiple sclerosis (PPMS) and secondary progressive multiple sclerosis (SPMS).

### BACKGROUND OF THE INVENTION

Copolymer-1, also known as glatiramer acetate (GA) and marketed under the tradename Copaxone®, comprises the acetate salts of random copolymers of four amino acids, namely L-glutamic acid, L-alanine, L-tyrosine and L-lysine. Glatiramer acetate is the acetate salt of a mixture of synthetic polypeptides, each of which consists essentially of the four naturally occurring amino acids: L-glutamic acid, L-alanine, L-tyrosine, and L-lysine with an average molar fraction of 0.141, 0.427, 0.095, and 0.338, respectively. The average molecular weight of glatiramer acetate is 5,000-9,000 Daltons. Glatiramer acetate is sold in USA as Copaxone®, indicated for reduction of the frequency of relapses in patients with Relapsing-Remitting Multiple Sclerosis (RRMS).

Copaxone® has been approved since 1996 for treating relapsing-remitting multiple sclerosis (RRMS) at a dose of 20 mg administered by daily subcutaneous injections. Since 2014, Copaxone® has also been approved at a dose of 40 mg administered by three injections per week, performed at least 48 hours apart. Compared to daily administration of Copaxone® in the 20 mg dose, the latter dose and regime reduce the yearly number of injections by about 200, while maintaining the same efficacy.

Daily and thrice-weekly Copaxone® treatments involve self-injection of the active substance. Frequently observed injection-site problems include irritation, hypersensitivity, inflammation, pain and even necrosis (in the case of interferon 1β treatment) and consequent problems in patient compliance. Side effects generally include a lump at the injection site (injection site reaction), aches, fever, and chills. These side effects are generally mild in nature. Occasionally a reaction occurs minutes after injection in which there is flushing, shortness in breath, anxiety and rapid heartbeat. These side effects subside within thirty minutes. Over time, a visible dent at the injection site due to the local destruction of fat tissue, known as lipoatrophy, may develop. Therefore, an alternative method of administration is desirable. Several serious side effects have been reported for glatiramer acetate, according to the FDA's prescribing label, these include serious side effects to the body's cardiovascular system, digestive system (including liver), hemic and lymphatic system, musculoskeletal system, nervous system, respiratory system, special senses (in particular the eyes), urogenital system; also reported have been metabolic and nutritional disorders; however a link between glatiramer acetate and these adverse effects has not been definitively established (FDA Copaxone® label).

The parenteral route by intravenous (IV), intramuscular (IM), or subcutaneous (SC) injection is the most common and effective form of delivery for small as well as large molecular weight drugs. However, pain, discomfort and inconvenience due to needle sticks makes this mode of drug delivery the least preferred by patients. Therefore, any drug delivery technology that can at a minimum reduce the total number of injections is preferred. Such reductions in frequency of drug dosing in practice may be achieved through the use of injectable depot formulations that are capable of releasing drugs in a slow but predictable manner and consequently improve compliance. For most drugs, depending on the dose, it may be possible to reduce the injection frequency from daily to once or twice monthly or even longer (6 months). In addition to improving patient comfort, less frequent injections of drugs in the form of depot formulations have been shown to reduce unwanted events, such as immunogenicity etc. often associated with large molecular weight drugs.

Microparticles, implants and gels are the most common forms of biodegradable polymeric devices used in practice for prolonging the release of drugs in the body. Microparticles are suspended in an aqueous media right before injection and one can load as much as 40% solids in suspensions. Implant/rod formulations are delivered to SC/IM tissue with the aid of special needles in the dry state without the need for an aqueous media. This feature of rods/implants allows for higher masses of formulation, as well as drug content to be delivered. Further, in the rods/implants, the initial burst problems are minimized due to much smaller area in implants compared to the microparticles. Besides biodegradable systems, there are non-biodegradable implants and infusion pumps that can be worn outside the body. Non-biodegradable implants require a doctor's visit not only for implanting the device into the SC/IM tissue but also to remove them after the drug release period.

Injectable compositions containing microparticle preparations are particularly susceptible to problems. Microparticle suspensions may contain as much as 40% solids as compared with 0.5-5% solids in other types of injectable suspensions. Further, microparticles used in injectable depot products, range in size up to about 250 μm (average, 60-100 μm), as compared with a particle size of less than 5 μm recommended for IM or SC administration. The higher concentrations of solids, as well as the larger solid particle size require larger size of needle (around 18-21 gauge) for injection. Overall, despite the infrequent uses of larger and uncomfortable needles, patients still prefer the considerably less frequently administered dosage forms over more frequent administration regimens such as every day or thrice weekly drug injections with a smaller needle.

Biodegradable polyesters of poly(lactic acid) (PLA) and copolymers of lactide and glycolide referred to as poly(lactide-co-glycolide) (PLGA) are the most common polymers used in biodegradable dosage forms. PLA is hydrophobic molecule and PLGA degrades faster than PLA because of the presence of more hydrophilic glycolide groups. These biocompatible polymers undergo random, non-enzymatic, hydrolytic cleavage of the ester linkages to form lactic acid and glycolic acid, which are normal metabolic compounds in the body. Resorbable sutures, clips and implants are the earliest applications of these polymers. Southern Research Institute developed the first synthetic, resorbable suture (Dexon®) in 1970. The first patent describing the use of PLGA polymers in a sustained release dosage form appeared in 1973 (U.S. Pat. No. 3,773,919).

Today, PLGA polymers are commercially available from multiple suppliers. Besides PLGA and PLA, natural cellulosic polymers such as starch, starch derivatives, dextran and non-PLGA synthetic polymers are also being explored as biodegradable polymers in such systems.

U.S. Pat. Nos. 8,377,885 and 8,796,226 to some of the present inventors relate to long acting pharmaceutical compositions comprising glatiramer acetate in depot form.

Ocrelizumab (OCREVUS™) is a humanized anti-CD20 monoclonal antibody, which was granted Breakthrough Therapy Designation for PPMS by the Food and Drug Administration (FDA) in 2016. It was approved by the FDA in 2017 as a treatment for multiple sclerosis, and it is the first FDA-approved drug for PPMS. It is administered by intravenous infusion.

To date, no long acting dosage forms of glatiramer acetate are commercially available for treating MS patients. An ongoing clinical trial is being conducted to test safety and/or efficacy of depot forms of GA in relapsing remitting forms of MS. There is an unmet medical need in treatment options for progressive forms of MS. Preferably, such treatments would be administered in depot formulations, to minimize the frequency of drug-delivery steps and associated side-effects.

### SUMMARY OF THE INVENTION

The present invention provides a method of treating progressive forms of multiple sclerosis (MS) and related symptoms, comprising administration or implantation of a long acting depot formulation of a pharmaceutically acceptable glatiramer salt, e.g., glatiramer acetate. According to various embodiments, the depot formulation is administered once every week, once every several weeks, once a month or once every several months. According to additional embodiments, the depot formulation provides a dose of 10-100 mg of pharmaceutically acceptable salts of glatiramer, e.g., glatiramer acetate, to a patient.

It is now disclosed for the first time that the long acting pharmaceutical compositions and depot formulations according to the principles of the present invention provide therapeutic efficacy in primary progressive multiple sclerosis (PPMS) and secondary progressive multiple sclerosis (SPMS) patients. It has further been unexpectedly found that administration once every several weeks of a depot of 10-100 mg glatiramer acetate according to the principles of the present invention is beneficial to PPMS and SPMS patients.

The present invention provides, in one aspect, a method for treating or alleviating primary progressive multiple sclerosis (PPMS), secondary progressive multiple sclerosis (SPMS) or at least one symptom of PPMS or SPMS, in a patient diagnosed with PPMS or SPMS, using depot formulations of GA. The method comprises the step of administering to the patient a therapeutically effective regimen of a depot formulation comprising glatiramer acetate (GA) or another pharmaceutically acceptable salt of glatiramer, the regimen being sufficient to treat or alleviate PPMS, SPMS or the at least one symptom of PPMS or SPMS. According to certain embodiments the depot formulation comprises glatiramer acetate.

In certain embodiments, the patient has been diagnosed as suffering from PPMS. In certain embodiments, the patient has been diagnosed as suffering from SPMS.

In certain embodiments, treating PPMS or SPMS comprises reducing the rate of progression of PPMS or SPMS. In certain embodiments, treating PPMS or SPMS comprises increasing the time to onset of Confirmed Disease Progression (CDP). In certain embodiments, treating PPMS or SPMS comprises increasing the time to onset of 12 week Confirmed Disease Progression (CDP) assessed by EDSS, compared to baseline. In certain embodiments, treating PPMS or SPMS comprises decreasing whole brain volume change or cortical volume change, compared to baseline. In certain embodiments, treating PPMS or SPMS comprises decreasing the time needed to complete a timed 25-foot walk (T25 FW) test, compared to baseline. In certain embodiments, treating PPMS or SPMS comprises decreasing the time needed to complete a 9-Hole Peg Test (9-HPT), compared to baseline.

In certain embodiments, treating PPMS or SPMS comprises decreasing (i) the number of new or enlarging T2 lesions; (ii) the volume of T2 lesions; (iii) the number of new or enlarging T1 lesions; (iv) the volume of T1 lesions; (v) the number or volume of Gadolinium (Gd) lesions; or (vi) any combination of (i) to (v). Each possibility represents a separate embodiment of the invention. In certain embodiments, treating PPMS or SPMS comprises preventing further progression of PPMS or SPMS, compared to baseline.

In certain embodiments, the symptom is selected from the group consisting of impaired coordination, impaired walking capability, impaired balance, weakness of the leg, stiffness of the leg, impaired memory, impaired cognitive function, a difficulty to swallow, impaired vision, general fatigue, pain, impaired bladder function, impaired bowel function, and any combination thereof. Each possibility represents a separate embodiment of the invention.

In certain embodiments, the depot formulation is administered once every 1 to 15 weeks. In certain embodiments, the depot formulation is administered once every 2 to 6 weeks. According to some embodiments, the administration comprises an injection of the depot formulation once every 2 to 6 weeks. In certain embodiments, the depot formulation is administered once every 4 weeks. According to some embodiments, the administration comprises intramuscular injection of the depot formulation once every 4 weeks.

In certain embodiments, the depot formulation is parenterally administered. In certain embodiments, the depot formulation is administered by intramuscular, subcutaneous, percutaneous, intravenous, or inhalation administration. Each possibility represents a separate embodiment of the invention. In certain embodiments, the depot formulation is intramuscularly administered. In certain embodiments, the depot formulation is subcutaneously administered.

In certain embodiments, the depot formulation is administered in a concentration of 20 mg GA per 1 mL of a carrier. In certain embodiments, the carrier is water for injection (WFI). In certain embodiments, the depot formulation comprises 20% to 30% solids. In certain embodiments, the depot formulation comprises a Poly(Lactide-co-Glycolide) (PLGA) copolymer. In certain embodiments, the PLGA copolymer is a poly(D,L-lactide-co-glycolide) (50:50) copolymer. In certain embodiments, the depot formulation comprises 550 mg PLGA copolymer per 40 mg of GA. In certain embodiments, the PLGA copolymer at least partly encapsulates the GA. In certain embodiments, the depot formulation comprises a 40 to 80 mg dose of GA. In certain embodiments, the depot formulation comprises a 40 mg dose of GA. In certain embodiments, the depot formulation comprises an 80 mg dose of GA.

In certain embodiments, less than 45% of the GA is released from the depot formulation within 14 days in PBS at 37° C. under continuous agitation. In certain embodiments, more than 90% of the GA is released from the depot formulation within 28 days in PBS at 37° C. under continuous agitation.

In certain embodiments, the patient (i) has been diagnosed with PPMS or SPMS for at least 1 year and a sustained increment of ≥1 point in the EDSS score in the last year or ≥0.5 points in EDSS score; (ii) has an EDSS score between 2 and 5.5, inclusive; (iii) has a documented history of, or the presence of more than 1 oligoclonal band (OCB) (IgG OCB positive (OCGB+)) and/or positive IgG index in the cerebrospinal fluid (CSF); (iv) has at least 1 gadolinium-enhancing lesion on MRI and/or at least 1 gadolinium-enhancing lesion documented within a previous year on MRI; or (v) any combination of (i) to (iv). Each possibility represents a separate embodiment of the invention. In certain embodiments, the patient has at least two separate areas of damage in the central nervous system (CNS) that have occurred at different points in time. In certain embodiments, the patient has a history of at least one year of disease progression, and at least two from the group consisting of (i) at least one area of damage in the CNS, (ii) at least two areas of damage of a similar type in the spinal cord, and (iii) oligoclonal band in the spinal fluid or an elevated IgG index. In certain embodiments, the patient has no history of relapse events. In certain embodiments, the patient has no history of remission events. In certain embodiments, the patient has not received GA therapy prior to initiation of the regimen of the present invention. In certain embodiments, the patient has received GA therapy prior to initiation of the regimen of the present invention. In certain embodiments, the patient has received daily GA therapy prior to initiation of the regimen of the present invention. In certain embodiments, the patient has received thrice weekly GA therapy prior to initiation of the regimen of the present invention.

In certain embodiments, a plurality of administrations of the depot formulations of the invention is provided to the subject in need thereof. In certain embodiments, the regimen is repeated at least twice. In certain embodiments, the regimen is consecutively repeated for at least 6 months. In certain embodiments, the regimen is consecutively repeated for at least 1 year.

In certain embodiments, the frequency of administration is reduced relative to daily administration of 20 mg GA or a thrice weekly administration of 40 mg GA. In certain embodiments, the dose of GA administrated is reduced relative to daily administration of 20 mg GA or a thrice weekly administration of 40 mg GA. Each possibility represents a separate embodiment of the invention.

The present invention further provides, in another aspect, a method of increasing the tolerability of a patient suffering from primary progressive multiple sclerosis (PPMS) or secondary progressive multiple sclerosis (SPMS) to GA treatment, the method comprising reducing the frequency of GA administration to a therapeutically effective regimen of a depot formulation of GA or another pharmaceutically acceptable salt of glatiramer, so as to thereby increase the tolerability of GA treatment in the patient.

In certain embodiments, increasing the tolerability of GA treatment comprises reducing the frequency of injections. In certain embodiments, increasing the tolerability of GA treatment comprises reducing the frequency of an injection site reaction. In certain embodiments, increasing the tolerability of GA treatment comprises reducing the total dose of GA administered over a period of time. In certain embodiments, increasing the tolerability of GA treatment comprises improving patient compliance.

The present invention further provides, in another aspect, a method of increasing the convenience of GA treatment of a patient suffering from primary progressive multiple sclerosis (PPMS) or secondary progressive multiple sclerosis (SPMS) by decreasing the administration frequency of GA. The method comprises reducing the frequency of administrations by instituting a therapeutically effective regimen of a depot formulation of GA or another pharmaceutically acceptable salt of glatiramer, so as to thereby increase the convenience of GA treatment of the patient.

The present invention further provides, in another aspect, a method of increasing the adherence to GA treatment of a patient suffering from primary progressive multiple sclerosis (PPMS) or secondary progressive multiple sclerosis (SPMS), the method comprising reducing the frequency of administrations of GA by instituting a therapeutically effective regimen of a depot formulation of GA or another pharmaceutically acceptable salt of glatiramer, so as to thereby increase the adherence to GA treatment of the patient.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 illustrates the mean clinical score results for saline control, Copaxone® (2 mg GA, days 0-8) and GA Depot (4 mg GA, day 0) in C57BL/6 mice groups. *P<0.05 for all treatment groups compared with untreated control, Single Factor ANOVA followed by one-tailed T Test assuming unequal variance. N=20/group, +/−standard error.

FIG. 2 illustrates the mean body weight results for saline control, Copaxone® (2 mg GA, days 0-8) and GA Depot (4 mg GA, day 0) in C57BL/6 mice groups. *P<0.05 for all treatment groups compared with untreated control, Single Factor ANOVA followed by one-tailed T Test assuming unequal variance. N=20/group, +/−standard error.

### DETAILED DESCRIPTION OF THE INVENTION

The present invention provides long acting depot formulations of glatiramer acetate (GA), with significant therapeutic efficacy in PPMS or SPMS patients, and reduced side effects resulting from infrequent GA administrations.

The invention is based on the surprising finding that GA, previously deemed ineffective in multiple clinical studies of PPMS patients, is indeed effective in treating PPMS and SPMS patients when formulated as depot systems and administered in lower-than-expected doses.

The present invention provides, in one aspect, a method for treating or alleviating PPMS or SPMS or at least one symptom of PPMS or SPMS in a patient diagnosed with PPMS or SPMS, the method comprising the step of administering to the patient a therapeutically effective regimen of a depot formulation comprising glatiramer acetate (GA) or another pharmaceutically acceptable salt of glatiramer, the regimen being sufficient to treat or alleviate PPMS or SPMS or the at least one symptom of PPMS or SPMS.

According to the principles of the present invention, the phrase “patient diagnosed as suffering from PPMS” or the term “PPMS patient” as used interchangeably herein refer to a patient diagnosed as suffering from PPMS, i.e. in a progressive phase of MS. According to the principles of the present invention, the phrase “patient diagnosed as suffering from SPMS” or the term “SPMS patient” as used interchangeably herein refer to a subject diagnosed as suffering from SPMS, i.e. after a relapsing-remitting phase has practically ended, after a progressive phase began, and in a progressive phase of MS.

In certain embodiments, the patient has been diagnosed as suffering from PPMS. In certain embodiments, the patient diagnosed as suffering from PPMS is in a progressive phase of PPMS. In certain embodiments, the patient has been diagnosed as suffering from SPMS. In certain embodiments, the patient diagnosed as suffering from SPMS is in a progressive phase of SPMS.

The term “therapeutically effective regimen” as used herein is intended to qualify the frequency of administration and the amount of GA that will achieve the goal of treatment or alleviation of PPMS or SPMS, or of treatment or alleviation of a symptom of PPMS or SPMS. The term “depot formulation” as used herein refers to a composition which provides prolonged, sustained or extended release of the glatiramer salt to the general systemic circulation of a subject or to local sites of action in a subject. This term may further refer to a composition which provides prolonged, sustained or extended duration of action (pharmacokinetics) of the glatiramer salt in a subject. The term “treating” as used herein refers to prevention, suppression or alleviation of a symptom or of a plurality of symptoms after the onset of PPMS or SPMS.

The term “glatiramer acetate” as used herein refers to a compound formerly known as Copolymer 1 that is sold under the trade name Copaxone® and consists of the acetate salts of synthetic polypeptides, containing four naturally occurring amino acids: L-glutamic acid, L-alanine, L-tyrosine, and L-lysine with an average molar fraction of 0.141, 0.427, 0.095, and 0.338, respectively. The average molecular weight of glatiramer acetate in Copaxone® is 4,700-11,000 daltons (FDA Copaxone® label) and the number of amino acid ranges between about 15 to about 100 amino acids. The term also refers to chemical derivatives and analogues of the compound. Typically the compound is prepared and characterized as specified in any of U.S. Pat. Nos. 5,981,589; 6,054,430; 6,342,476; 6,362,161; 6,620,847; and 6,939,539, the contents of each of these references are hereby incorporated in their entirety.

The copolymers can be made by any procedure available to one of skill in the art. For example, the copolymers can be made under condensation conditions using the desired molar ratio of amino acids in solution, or by solid phase synthetic procedures. Condensation conditions include the proper temperature, pH, and solvent conditions for condensing the carboxyl group of one amino acid with the amino group of another amino acid to form a peptide bond. Condensing agents, for example, dicyclohexylcarbodiimide, can be used to facilitate the formation of the peptide bond.

In some embodiments, the composition may comprise any other pharmaceutically acceptable salt of glatiramer including, but not limited to, sulfate, pyrosulfate, bisulfate, sulfite, bisulfate, phosphate, monohydrogenphosphate, dihydrogenphosphate, metaphosphate, pyrophosphate, hydrochloride, hydrobromide, hydroiodide, acetate, nitrate, propionate, decanoate, caprylate, acrylate, formate, isobutyrate, caprate, heptanoate, propiolate, oxalate, malonate, succinate, tocopheryl succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylenesulfonate, phenylacetate, phenylpropionate, phenylbutyrate, citrate, lactate, β-hydroxybutyrate, glycollate, tartrate, methanesulfonate, propanesulfonate, naphthalene-2-sulfonate, p-toluenesulfonate, mandelate and the like salts. Each possibility represents a separate embodiment of the invention.

In certain embodiments, the symptom is impaired coordination. In certain embodiments, the symptom is impaired walking capability. In certain embodiments, the symptom is impaired balance. In certain embodiments, the symptom is weakness of the leg. In certain embodiments, the symptom is stiffness of the leg. In certain embodiments, the symptom is impaired memory. In certain embodiments, the symptom is impaired cognitive function. In certain embodiments, the symptom is a difficulty to swallow. In certain embodiments, the symptom is impaired vision. In certain embodiments, the symptom is general fatigue. In certain embodiments, the symptom is pain. In certain embodiments, the symptom is impaired bladder function. In certain embodiments, the symptom is impaired bowel function. In certain embodiments, the symptom is a combination of PPMS or SPMS symptoms.

In certain embodiments, the depot formulation is administered once every 1 to 15 weeks. In certain embodiments, the depot formulation is administered once every 1 to 10 weeks. According to some embodiments, the administration comprises an injection. According to some embodiments, the administration comprises an injection every 2 to 6 weeks. According to some embodiments, the administration comprises an administration every 2 to 6 weeks. In certain embodiments, the depot formulation is administered once every 2 weeks. In certain embodiments, the depot formulation is administered once every 3 weeks. In certain embodiments, the depot formulation is administered once every 4 weeks. In certain embodiments, the depot formulation is administered once every 5 weeks. In certain embodiments, the depot formulation is administered once every 6 weeks. In certain embodiments, the depot formulation is parenterally administered. In certain embodiments, the depot formulation is administered by intramuscular, subcutaneous, percutaneous, intravenous, or inhalation administration. Each possibility represents a separate embodiment of the invention. In certain embodiments, the depot formulation is intramuscularly administered. In certain embodiments, the depot formulation is subcutaneously administered.

In certain embodiments, the depot formulation is administered in a concentration of 10 mg GA per 1 mL of a carrier. In certain embodiments, the depot formulation is administered in a concentration of 20 mg GA per 1 mL of a carrier. In certain embodiments, the depot formulation is administered in a concentration of 40 mg GA per 1 mL of a carrier. In certain embodiments, the carrier is WFI. The term “water for injection” or “WFI” as used herein generally means sterile, pure water that meets regulatory standards for e.g. particulates, dissolved solids, organics, inorganics, microbial and endotoxin contaminants. In certain embodiments, the depot formulation is administered in WFI) or a buffer containing a suspending agent (e.g. carboxymethylcellulose, CMC), a buffering agent (e.g. citrate salts) and/or a tonicity agent (e.g. NaCl).

In certain embodiments, the depot formulation comprises 10% to 40% solids. In certain embodiments, the depot formulation comprises 20% to 30% solids. In certain embodiments, the depot formulation comprises a Poly(Lactide-co-Glycolide) (PLGA) copolymer. In certain embodiments, the PLGA copolymer is a poly(D,L-lactide-co-glycolide) (50:50) copolymer. In certain embodiments, the depot formulation comprises 150-1500 mg PLGA copolymer per 40 mg of GA. In certain embodiments, the depot formulation comprises 550 mg PLGA copolymer per 40 mg of GA. In certain embodiments, the PLGA copolymer at least partly encapsulates the GA. In certain embodiments, the PLGA copolymer encapsulates the GA.

In certain embodiments, the depot formulation comprises at least 20 mg dose of GA or of another pharmaceutically acceptable salt thereof. In certain embodiments, the depot formulation comprises between 20 to 1000 mg dose of GA or of another pharmaceutically acceptable salt thereof. In certain embodiments, the depot formulation comprises between 20 to 750 mg dose of GA or of another pharmaceutically acceptable salt thereof. In certain embodiments, the depot formulation comprises 40 to 80 mg dose of GA. In certain embodiments, the depot formulation comprises a 40 mg dose of GA. In certain embodiments, the depot formulation comprises a 50 mg dose of GA. In certain embodiments, the depot formulation comprises a 60 mg dose of GA. In certain embodiments, the depot formulation comprises a 70 mg dose of GA. In certain embodiments, the depot formulation comprises an 80 mg dose of GA.

In certain embodiments, less than 30% of the GA is released from the depot formulation within 7 days in PBS at 37° C. under continuous agitation. In certain embodiments, more than 20% of the GA is released from the depot formulation within 7 days in PBS at 37° C. under continuous agitation. In certain embodiments, less than 45% of the GA is released from the depot formulation within 14 days in PBS at 37° C. under continuous agitation. In certain embodiments, more than 30% of the GA is released from the depot formulation within 14 days in PBS at 37° C. under continuous agitation. In certain embodiments, less than 85% of the GA is released from the depot formulation within 21 days in PBS at 37° C. under continuous agitation. In certain embodiments, more than 40% of the GA is released from the depot formulation within 21 days in PBS at 37° C. under continuous agitation. In certain embodiments, more than 90% of the GA is released from the depot formulation within 28 days in PBS at 37° C. under continuous agitation.

In certain embodiments, in PBS at 37° C. under continuous agitation, (i) about 14% of the glatiramer is released from the depot formulation within 0 days, and/or (ii) about 15% of the glatiramer is released from the depot formulation within 1 day, and/or (iii) about 21% of the glatiramer is released from the depot formulation within 5 days, and/or (iv) about 25% of the glatiramer is released from the depot formulation within 8 days, and/or (v) about 34% of the glatiramer is released from the depot formulation within 13 days, and/or (vi) about 43% of the glatiramer is released from the depot formulation within 15 days, and/or (vii) about 80% of the glatiramer is released from the depot formulation within 22 days, and/or (viii) about 96% of the glatiramer is released from the depot formulation within 27 days, and/or (ix) about 99% of the glatiramer is released from the depot formulation within 32 days. Each possibility and each combination of possibilities represents a separate embodiment of the invention.

In certain embodiments, treating PPMS or SPMS comprises reducing the rate of progression of a PPMS- or SPMS-related symptom. In certain embodiments, treating PPMS or SPMS comprises reducing the rate of progression of PPMS or SPMS. In certain embodiments, treating PPMS or SPMS comprises increasing the time to onset of Confirmed Disease Progression (CDP). In certain embodiments, treating PPMS or SPMS comprises increasing the time to onset of 12 week Confirmed Disease Progression (CDP) assessed by EDSS, compared to baseline. The Kurtzke Expanded Disability Status Scale (EDSS) is a method of quantifying disability in multiple sclerosis. The EDSS quantifies disability in eight Functional Systems (FS) and allows neurologists to assign a Functional System Score (FSS) in each of these. The EDSS measures disability status on a scale ranging from 0 to 10, with higher scores indicating more disability. In certain embodiments, treating PPMS or SPMS comprises decreasing whole brain volume change or cortical volume change, compared to baseline. The T25FW is a quantitative mobility and leg function performance test where the participant is timed while walking for 25 feet. In certain embodiments, treating PPMS or SPMS comprises decreasing the time needed to complete a timed 25-foot walk (T25FW) test, compared to baseline. The 9-HPT is a quantitative test of upper extremity function that measures the time it takes to place 9 pegs into 9 holes and then remove the pegs. In certain embodiments, treating PPMS comprises decreasing the time needed to complete a 9-Hole Peg Test (9-HPT), compared to baseline. The terms “baseline” and “control” are interchangeable, and used herein to refer to a period of time before imitating treatment by the method of the present invention. In certain embodiments, the term “baseline” as used herein further refers to PPMS or SPMS patients which are untreated by the method of the present invention. In certain embodiments, the term “baseline” as used herein refers to a period of 1 year before imitating using the treatment by the method of the present invention.

In certain embodiments, CDP is defined as one or more of the following criteria, confirmed using one or more of the following assessments: sustained EDSS score increased from baseline of ≥1 point if baseline EDSS≤5.5, or ≥0.5 point if Baseline EDSS>5.5. In certain embodiments, CDP is defined as a sustained (≥12 weeks) increase in EDSS from baseline of ≥1.0 points if the baseline EDSS was between 2.0 and 5.5 points or an EDSS increase of ≥0.5 points if the baseline EDSS was >5.5 points.

In certain embodiments, treating PPMS or SPMS comprises decreasing the number of new or enlarging T2 lesions. In certain embodiments, treating PPMS or SPMS comprises decreasing the volume of T2 lesions. In certain embodiments, treating PPMS or SPMS comprises decreasing the number of new or enlarging T1 lesions. In certain embodiments, treating PPMS or SPMS comprises decreasing the volume of T1 lesions. In certain embodiments, treating PPMS or SPMS comprises decreasing the number or volume of Gadolinium (Gd) lesions. In certain embodiments, treating PPMS or SPMS comprises preventing further progression of PPMS or SPMS, compared to baseline.

In certain embodiments, the patient has been diagnosed with PPMS or SPMS for at least 1 year and a sustained increment of ≥1 point in the EDSS score in the last year. In certain embodiments, the patient has an EDSS score between 2 and 5.5, inclusive. In certain embodiments, the patient has a documented history of, or the presence of more than 1 oligoclonal band (OCB) (IgG OCB positive (OCGB+)) and/or positive IgG index in the cerebrospinal fluid (CSF). In certain embodiments, the patient has at least 1 gadolinium-enhancing lesion on MRI and/or at least 1 gadolinium-enhancing lesion documented within a previous year on MRI. In certain embodiments, the patient has at least two separate areas of damage in the central nervous system (CNS) that have occurred at different points in time. In certain embodiments, the patient has a history of at least one year of disease progression, and at least two from the group consisting of (i) at least one area of damage in the CNS, (ii) at least two areas of damage of a similar type in the spinal cord, and (iii) oligoclonal band in the spinal fluid or an elevated IgG index. Each possibility represents a separate embodiment of the invention. In certain embodiments, the patient has no history of relapse events. In certain embodiments, the patient has no history of remission events. In certain embodiments, the patient has not received GA therapy prior to initiation of the regimen. In certain embodiments, the patient has received GA therapy prior to initiation of the regimen.

In certain embodiments, the regimen is repeated. In certain embodiments, the regimen is repeated at least twice. In certain embodiments, the regimen is consecutively repeated for at least 6 months. In certain embodiments, the regimen is consecutively repeated for at least 1 year. In certain embodiments, the regimen is consecutively repeated for the life time of the patient.

In certain embodiments, the frequency of administration is reduced relative to daily administration of 20 mg GA or a thrice weekly administration of 40 mg GA. In certain embodiments, the dose of GA administrated is reduced relative to daily administration of 20 mg GA or a thrice weekly administration of 40 mg GA. Each possibility represents a separate embodiment of the invention. In some embodiments, the compositions of the present invention provide prolonged release or prolonged action of glatiramer in a subject as compared to a substantially similar dose of an immediate release formulation of glatiramer acetate.

The present invention further provides, in another aspect, a method of increasing the tolerability of a patient suffering from primary progressive multiple sclerosis (PPMS) or secondary progressive multiple sclerosis (SPMS) to GA treatment, the method comprising reducing the frequency of GA administration from daily subcutaneous injections of a 20 mg dose of GA or three subcutaneous injections of a 40 mg dose of GA over a period of seven days with at least one day between every injection, to a therapeutically effective regimen of a depot formulation of GA or another pharmaceutically acceptable salt of glatiramer, so as to thereby increase the tolerability of GA treatment in the patient.

In certain embodiments, increasing the tolerability of GA treatment comprises reducing the frequency of injections. In certain embodiments, increasing the tolerability of GA treatment comprises reducing the frequency of an injection site reaction.

The present invention further provides, in another aspect, a method of increasing the convenience of GA treatment of a patient suffering from primary progressive multiple sclerosis (PPMS) or secondary progressive multiple sclerosis (SPMS), the method comprising reducing the frequency of GA administration to a therapeutically effective regimen of a depot formulation of GA or another pharmaceutically acceptable salt of glatiramer, so as to thereby increase the convenience of GA treatment of the patient.

The present invention further provides, in another aspect, a method of increasing the adherence to GA treatment of a patient suffering from primary progressive multiple sclerosis (PPMS) or secondary progressive multiple sclerosis (SPMS), the method comprising reducing the frequency of GA administration to a therapeutically effective regimen of a depot formulation of GA or another pharmaceutically acceptable salt of glatiramer, so as to thereby increase the adherence to GA treatment of the patient.

The present invention further provides, in another aspect, a depot formulation comprising glatiramer acetate (GA) or another pharmaceutically acceptable salt of glatiramer for use in a method for treating or alleviating PPMS, SPMS or at least one symptom of PPMS or SPMS in a patient diagnosed with PPMS or SPMS, the method comprising the step of administering the depot formulation to the patient.

The present invention further provides, in another aspect, a depot formulation comprising glatiramer acetate (GA) or another pharmaceutically acceptable salt of glatiramer for use in a method of increasing the tolerability of a patient suffering from primary progressive multiple sclerosis (PPMS) or secondary progressive multiple sclerosis (SPMS) to GA treatment, the method comprising reducing the frequency of GA administration from daily subcutaneous injections of a 20 mg dose of GA or three subcutaneous injections of a 40 mg dose of GA over a period of seven days with at least one day between every injection, to an administration of the depot formulation.

The present invention further provides, in another aspect, a depot formulation comprising glatiramer acetate (GA) or another pharmaceutically acceptable salt of glatiramer for use in a method of increasing the convenience of GA treatment of a patient suffering from primary progressive multiple sclerosis (PPMS) or secondary progressive multiple sclerosis (SPMS), the method comprising reducing the frequency of GA administration to a therapeutically effective regimen of the depot formulation.

The present invention further provides, in another aspect, a depot formulation comprising glatiramer acetate (GA) or another pharmaceutically acceptable salt of glatiramer for use in a method of increase the adherence to GA treatment of a patient suffering from primary progressive multiple sclerosis (PPMS) or secondary progressive multiple sclerosis (SPMS), the method comprising reducing the frequency of GA administration to a therapeutically effective regimen of the depot formulation.

In certain embodiments, dosage forms include, but are not limited to, biodegradable injectable depot systems such as, PLGA based injectable depot systems; non-PLGA based injectable depot systems, and injectable biodegradable gels or dispersions. Each possibility represents a separate embodiment of the invention. The term “biodegradable” as used herein refers to a component which erodes or degrades at its surfaces over time due, at least in part, to contact with substances found in the surrounding tissue fluids, or by cellular action. In particular, the biodegradable component is a polymer such as, but not limited to, lactic acid-based polymers such as polylactides e.g. poly(D,L-lactide) i.e. PLA; glycolic acid-based polymers such as polyglycolides (PGA) e.g. Lactel® from Durect; poly(D,L-lactide-co-glycolide) i.e. PLGA, (Resomer® RG-504, Resomer® RG-502, Resomer® RG-504H, Resomer® RG-502H, Resomer® RG-504S, Resomer® RG-502S, from Boehringer, Lactel® from Durect); polycaprolactones such as Poly(e-caprolactone) i.e. PCL (Lactel® from Durect); polyanhydrides; poly(sebacic acid) SA; poly(ricenolic acid) RA; poly(fumaric acid), FA; poly(fatty acid dimmer), FAD; poly(terephthalic acid), TA; poly(isophthalic acid), IPA; poly(p-{carboxyphenoxy}methane), CPM; poly(p-{carboxyphenoxy}propane), CPP; poly(p-{carboxyphenoxy}hexane) s CPH; polyamines, polyurethanes, polyesteramides, polyoithoesters {CHDM: cis/trans-cyclohexyl dimethanol, HD: 1,6-hexanediol. DETOU: (3,9-diethylidene-2,4,8,10-tetraoxaspiro undecane)}; polydioxanones; polyhydroxybutyrates; polyalkylene oxalates; polyamides; polyesteramides; polyurethanes; polyacetals; polyketals; polycarbonates; polyorthocarbonates; polysiloxanes; polyphosphazenes; succinates; hyaluronic acid; poly(malic acid); poly(amino acids); polyhydroxyvalerates; polyalkylene succinates; polyvinylpyrrolidone; polystyrene; synthetic cellulose esters; polyacrylic acids; polybutyric acid; triblock copolymers (PLGA-PEG-PLGA), triblock copolymers (PEG-PLGA-PEG), poly(N-isopropylacrylamide) (PNIPAAm), poly(ethylene oxide)-poly (propylene oxide)-poly(ethylene oxide) tri-block copolymers (PEO-PPO-PEO), poly valeric acid; polyethylene glycol; polyhydroxyalkylcellulose; chitin; chitosan; polyorthoesters and copolymers, terpolymers; lipids such as cholesterol, lecithin; poly(glutamic acid-co-ethyl glutamate) and the like, or mixtures thereof.

In some embodiments, the compositions of the present invention comprise a biodegradable polymer selected from, but not limited to, PLGA, PLA, PGA, polycaprolactone, polyhydroxybutyrate, polyorthoesters, polyalkaneanhydrides, gelatin, collagen, oxidized cellulose, polyphosphazene and the like. Each possibility represents a separate embodiment.

In certain embodiments, the biodegradable polymer is a lactic acid-based polymer, more preferably polylactide, or poly(D, L-lactide-co-glycolide) i.e. PLGA. The biodegradable polymer is present in an amount between about 10% to about 98% w/w of the composition. The lactic acid-based polymer has a monomer ratio of lactic acid to glycolic acid in the range of 100:0 to about 0:100, preferably 100:0 to about 10:90 and has an average molecular weight of from about 1,000 to 200,000 Daltons. However, it is understood that the amount of biodegradable polymer is determined by parameters such as the duration of use and the like.

The compositions and formulations of the present invention may further comprise one or more pharmaceutically acceptable excipient(s) selected from, but not limited to, co-surfactants, solvents/co-solvents, water immiscible solvents, water, water miscible solvents, oily components, hydrophilic solvents, emulsifiers, preservatives, antioxidants, anti-foaming agents, stabilizers, buffering agents, pH adjusting agents, osmotic agents, channel forming agents, osmotic adjustment agents, or any other excipient known in the art. Suitable co-surfactants include, but are not limited to, polyethylene glycols, polyoxyethylene-polyoxypropylene block copolymers known as “poloxamer”, polyglycerin fatty acid esters such as decaglyceryl monolaurate and decaglyceryl monomyristate, sorbitan fatty acid ester such as sorbitan monostearate, polyoxyethylene sorbitan fatty acid ester such as polyoxyethylene sorbitan monooleate (Tween), polyethylene glycol fatty acid ester such as polyoxyethylene monostearate, polyoxyethylene alkyl ether such as polyoxyethylene lauryl ether, polyoxyethylene castor oil and hardened castor oil such as polyoxyethylene hardened castor oil, and the like or mixtures thereof. Each possibility represents a separate embodiment of the invention. Suitable solvents/co-solvents include, but not limited to, alcohols, triacetin, dimethyl isosorbide, glycofurol, propylene carbonate, water, dimethyl acetamide, and the like or mixtures thereof. Each possibility represents a separate embodiment of the invention. Suitable anti-foaming agents include, but are not limited to, silicon emulsions or sorbitan sesquioleate. Suitable stabilizers to prevent or reduce the deterioration of the components in the compositions of the present invention include, but are not limited to, antioxidants such as glycine, a-tocopherol or ascorbate, BHA, BHT, and the like or mixtures thereof. Each possibility represents a separate embodiment of the invention. Suitable tonicity modifiers include, but are not limited to, mannitol, sodium chloride, and glucose. Each possibility represents a separate embodiment of the invention. Suitable buffering agents include, but are not limited to, acetates, phosphates, and citrates with suitable cations. Each possibility represents a separate embodiment of the invention.

The particle size of the “water-in oil-in water (w/o/w) double emulsion” can be determined by various parameters including, but not limited to, the amount of applied force at this step, the speed of mixing, surfactant type and concentration, etc. Suitable particle sizes range from about 1 to 100 μm.

The depot systems of the present invention encompass any forms known to a person of skill in the art. Suitable forms include, but are not limited to, biodegradable or non-biodegradable microspheres, implantable rods, implantable capsules, and implantable rings. Each possibility represents a separate embodiment of the invention. Further contemplated are prolonged release gel depot and erodible matrices. Each possibility represents a separate embodiment of the invention. Suitable implantable systems are described for example in US 2008/0063687, the content of which is hereby incorporated in its entirety. Implantable rods can be prepared as is known in the art using suitable micro-extruders.

In some embodiments, the depot formulations of the present invention include, but are not limited to, suspensions of glatiramer acetate in water, oil or wax phase; poorly soluble polyelectrolyte complexes of glatiramer acetate; “in-situ” gel-forming matrices based on the combination of water-miscible solvent with glatiramer acetate; and biodegradable polymeric microparticles with incorporated glatiramer acetate. Each possibility represents a separate embodiment of the invention. In particular, the compositions of the present invention are in the form of injectable microparticles wherein the glatiramer acetate is entrapped in a biodegradable or non-biodegradable carrier. The microparticulate compositions of the present invention may comprise a water-in oil-in water double emulsion. Within the scope of the present invention is a microparticulate composition comprising an internal aqueous phase comprising glatiramer or any pharmaceutically acceptable salt thereof, an oil phase or water-immiscible phase comprising a biodegradable or non-biodegradable polymer and an external aqueous phase. The external aqueous phase may further comprise a surfactant, preferably polyvinyl alcohol (PVA), polysorbate, polyethylene oxide-polypropylene oxide block copolymers or cellulose esters. The terms “oil phase” and “water-immiscible phase” may be used interchangeably herein.

According to some embodiments, the glatiramer acetate comprises the acetate salt of L-alanine, L-glutamic acid, L-lysine, and L-tyrosine in the molar ratios of about 0.14 glutamic acid, about 0.43 alanine, about 0.10 tyrosine and about 0.33 lysine. According to other embodiments, the glatiramer acetate or other pharmaceutically acceptable salt of glatiramer comprises about 15 to about 100 amino acids.

In certain embodiments, the depot formulation is self-administered intramuscularly by the patient. In certain embodiments, the depot formulation is injected into the deltoid muscle.

Encompassed by the present invention is a combination therapy of glatiramer acetate or any other pharmaceutically acceptable salt of glatiramer with at least one other active agent. Active agents within the scope of the present invention include, but are not limited to interferons, e.g. pegylated or non-pegylated α-interferons, or β-interferons, e.g. interferon β-1a or interferon β-1b, or τ-interferons; immuno-suppressants with optionally antiproliferative/antineoplastic activity, e.g. mitoxantrone, methotrexate, azathioprine, cyclophosphamide, or steroids, e.g. methylprednisolone, prednisone or dexamethasone, or steroid-secreting agents, e.g. ACTH; adenosine deaminase inhibitors, e.g. cladribine; IV immunoglobulin G (e.g. as disclosed in Neurology, 1998 May 50(5): 1273-81) monoclonal antibodies to various T-cell surface markers, e.g. natalizumab (ANTEGREN®) or alemtuzumab; TH2 promoting cytokines, e.g. IL-4, IL-10, or compounds which inhibit expression of TH1 promoting cytokines, e.g. phosphodiesterase inhibitors, e.g. pentoxifylline; antispasticity agents including baclofen, diazepam, piracetam, dantrolene, lamotrigine, rifluzole, tizanidine, clonidine, beta blockers, cyproheptadine, orphenadrine or cannabinoids; AMPA glutamate receptor antagonists, e.g. 2,3-dihydroxy-6-nitro-7-sulfamoylbenzo(f)quinoxaline, [1,2,3,4,-tetrahydro-7-morpholin-yl-2,3-dioxo-6-(trifluoromethyl)quinoxalin-1-yl]methylphosphonate, 1-(4-aminophenyl)-4-methyl-7,8-methylene-dioxy-5H-2,3-benzodiazepine, or (−)1-(4-aminophenyl)-4-methyl-7,8-methylene-dioxy-4,5-dihydro-3-methylcarbarnoyl-2,3-benzodiazepine; inhibitors of VCAM-1 expression or antagonists of its ligand, e.g. antagonists of the α4β1 integrin VLA-4 and/or α-4-β-7 integrins, e.g. natalizumab (ANTEGREN®); anti-macrophage migration inhibitory factor (Anti-MIF); xii) Cathepsin S inhibitors; xiii) mTor inhibitors. Each possibility represents a separate embodiment of the invention. Currently preferred one other active agent is FTY720 (2-amino-2-[2-(4-octylphenyl)ethyl]propane-1,3-diol; fingolimod) belonging to the class of immuno-suppressants. Another possibility is to combine glatiramer acetate with treatment by ocrelizumab (OCREVUS™), a humanized anti-CD20 monoclonal antibody, which is the only therapy currently approved for treatment of PPMS.

In more specific embodiments, the sustained release depot formulation comprises a therapeutically effective amount of a pharmaceutically acceptable salt of glatiramer, the formulation being in a sustained release depot form which releases a therapeutically effective amount of the pharmaceutically acceptable salt of glatiramer over a period of about one week to about 6 months. In certain embodiments, the sustained release depot formulation comprises a therapeutically effective amount of a pharmaceutically acceptable salt of glatiramer in depot form suitable for implantation at a medically acceptable location in a subject in need thereof. In certain embodiments, the glatiramer comprises L-alanine, L-glutamic acid, L-lysine, and L-tyrosine in molar ratios of about 0.14 glutamic acid, about 0.43 alanine, about 0.10 tyrosine and about 0.33 lysine. In certain embodiments, the glatiramer comprises about 15 to about 100 amino acids. In certain embodiments, the sustained release depot formulation further comprises a pharmaceutically acceptable biodegradable or non-biodegradable carrier. In certain embodiments, the carrier is selected from poly(D,L-lactide-co-glycolide) (PLGA), poly (D,L-lactide) (PLA), polyglycolides (PGA), polycaprolactone, polyhydroxybutyrate, polyorthoesters, polyalkaneanhydrides, gelatin, collagen, oxidized cellulose, and polyphosphazene. In certain embodiments, the sustained release depot formulation is in the form of microparticles prepared by a water-in oil-in water double emulsification process. In certain embodiments, the sustained release depot formulation comprises an internal aqueous phase comprising a therapeutically effective amount of a pharmaceutically acceptable salt of glatiramer, a water immiscible polymeric phase comprising a biodegradable or non-biodegradable polymer and an external aqueous phase. In certain embodiments, the water immiscible polymeric phase comprises a biodegradable polymer selected from poly(D,L-lactide) (PLA) and poly(D,L-lactide-co-glycolide) (PLGA). In certain embodiments, the external water phase comprises a surfactant selected form polyvinyl alcohol (PVA), polysorbate, polyethylene oxide-polypropylene oxide block copolymers and cellulose esters. In certain embodiments, the sustained release depot formulation is in the form of biodegradable microspheres, non-biodegradable microspheres, implants of any suitable geometric shape, implantable rods, implantable capsules, implantable rings, or prolonged release gels or erodible matrices. In certain embodiments, the sustained release depot formulation provides equal or superior therapeutic efficacy to the commercially available daily or thrice weekly injectable dosage forms of glatiramer acetate, with reduced incidence of side effects and/or with reduced severity of side effects at the local and/or systemic level. In certain embodiments, the sustained release depot formulation provides prolonged release or prolonged action of glatiramer in a subject as compared to a substantially similar dose of an immediate release formulation of glatiramer acetate.

In certain embodiments, the sustained release depot formulation further comprises at least one additional drug. In certain embodiments, the at least one additional drug is an immunosuppressant. In certain embodiments, the at least one additional drug is fingolimod. In certain embodiments, the sustained release depot formulation comprises the pharmaceutically acceptable salt of glatiramer in a dose ranging from about 20 to about 750 mg. In certain embodiments, the pharmaceutically acceptable salt of glatiramer is selected from the group consisting of sulfate, pyrosulfate, bisulfate, sulfite, bisulfite, phosphate, monohydrogenphosphate, dihydrogenphosphate, metaphosphate, pyrophosphate, hydrochloride, hydrobromide, hydroiodide, acetate, nitrate, propionate, decanoate, caprylate, acrylate, formate, isobutyrate, caprate, heptanoate, propiolate, oxalate, malonate, succinate, tocopheryl succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylenesulfonate, phenylacetate, phenylpropionate, phenylbutyrate, citrate, lactate, .beta.-hydroxybutyrate, glycollate, tartrate, methanesulfonate, propanesulfonate, naphthalene-2-sulfonate, p-toluenesulfonate, and mandelate. Each possibility represents a separate embodiment of the present invention. In certain embodiments, the therapeutically acceptable amount of said pharmaceutically acceptable salt of glatiramer is about 1 to about 500 mg/day, or about 20 mg to about 200 mg/day.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### Examples

### Example 1: In-Vitro Preparation Method of 40 mg GA in PLGA Depot.

Preparation Process: (1) External water phase preparation: Partially hydrolyzed polyvinyl alcohol (PVA) solution at a concentration of 2% w/w in sterile WFI was prepared in a reactor and filtered through a 0.22 μm membrane. (2) A solution of NaCl in sterile WFI was prepared and filtered through a 0.22 μm membrane into the reactor containing the PVA. (3) Organic phase preparation: Organic phase composed of dichloromethane and poly(lactide-co-glycolide) was prepared in a reactor and filtered through a 0.22 μm membrane. (4) Internal water phase preparation: A solution containing sterile WFI and glatiramer acetate was prepared and filtered through a 0.22 μm membrane. (5) Water-in-oil (w/o) emulsion preparation: Internal water phase was added to the organic phase and processed using IKA Ultra-Turrax T50 homogenizer equipped with a rotor stator dispersion device at 7,200 RPM for 10 minutes (high shear mixing). (6) Water-in-oil-in-water (w/o/w) emulsion preparation: Water in oil emulsion (w/o) prepared in step 5 was added to half of the external water phase during continuing mixing of the w/o emulsion. The w/o/w double emulsion was processed using IKA Ultra-Turrax UTS80 homogenizer with a rotor stator head at 2,900 RPM for 3 minutes from the end of w/o transfer into the external water phase. Following, another 30 liters of the external water phase was added to the emulsion (quench). (7) Solvent removal/evaporation: The w/o/w double emulsion formed in step (6) was mixed using the IKA UTS80 homogenizer at different speeds for 15-17 hours. Compressed air was bubbled at 0.5 Pa through the emulsion for 10-12 hours. Vacuum was applied for the portion of the process. (8) Separation and washing: The suspension was centrifuged at 5,300 RPM for 10 minutes. The supernatant was discarded and the pellet (sediment microparticles) is resuspended in 550 g WFI and mixed using a magnetic stirrer for 3 minutes. The resuspended microparticles were centrifuged at 2900 RPM for 10 minutes. (9) Lyophilization: The washed microparticles were resuspended in about 750 g sterile WFI and are kept at −20° C. until lyophilization. Lyophilization was carried out using sterile lyoguard trays as follows: Freeze at −40° C., 24 hours. Primary drying at 0.2 hPa, −5° C., 48 hours. Secondary drying at 0.2 hPa, 10° C., 48 hours. The resulting composition includes GA and PLGA (50:50, molecular weight 7,000-17,000) in a 1:11.5 weight ratio.

Example 2: In-Vitro Release Profile of 40 mg GA from a PLGA Depot.

The release of the incorporated glatiramer acetate was carried out in tightly closed 20 ml glass vials, using incubator at 37° C., equipped with a multi-point magnetic stirrer. Phosphate buffered saline (PBS) with pH 7.4 was used as a release media. Table 1 summarizes the release profile of GA from a PLGA Depot.

TABLE 1

| | Days | | | | | | | | |
|---|---|---|---|---|---|---|---|---|---|
| | 0 | 1 | 5 | 8 | 13 | 15 | 22 | 27 | 32 |
| % GA released into medium | 14 | 15 | 21 | 25 | 34 | 43 | 80 | 96 | 99 |

Example 3: Depot of 40 mg GA Vs. Commercially Available GA (Copaxone®) In-Vivo.

### Material and Methods

Animals: All animal studies were approved by the Israeli MOH Animal Care and Use Committee. C57BL/6 female mice, 7-9 weeks old were randomized into control or treatment groups with similar mean weight. Animals were given food and water ad-libitum throughout the experiment.
Induction of progressive EAE: In order to induce primary progressive EAE (PP-EAE) which most resembles progressive forms of MS in humans (Sayed et al., The Journal of Immunology, 2011, Vol. 186, 3294-3298), an emulsion of MOG 35-55 (GL Biochem co. Ltd, Shanghai, China) in Modified Complete Freund's adjuvant (CFA) (Sigma-Aldrich, St. Louis, MO, USA) was prepared as follows: heat-killed M. tuberculosis Strain H37RA (Sigma) was added to CFA to a final concentration of 4 mg/mL. Subsequently, 2 mg/mL MOG 35-55 were emulsified with equal amount of modified CFA. EAE had been induced by injection of this emulsion subcutaneously (SC) on the shaved back of the mouse at one site, followed by an intraperitoneal injection of Bordetella pertussis toxin (Sigma) in PBS on Day 0 and 48 hours post MOG immunization. A 21 G needle was used for injections in mice. This model displays a robust inflammation stage on days 9 to 15 in which demyelinization as well as axonal damage is evident. Following this stage, inflammation is usually very moderate.
Measurements: Body weight was measured every two days from day 0 to day 28. EAE was assessed by clinical scoring of the mice once daily from Day 0 to Day 28 post immunization (Table 2). For analysis, dead animals received clinical score of 5 and the weight recorded at the last measurement before animal death.

TABLE 2
EAE clinical Score in Mice.

| Score | Clinical signs |
|---|---|
| 0 | Normal mouse; no overt signs of disease |
| 1 | Limp tail |
| 2 | Hind limb paralysis |
| 3 | Hind and front limb paralysis |
| 4 | Complete paralysis: sacrifice for humane reasons |
| 5 | Moribund state; Death by EAE |

The following calculations were derived from clinical score raw data: mean maximum score is the mean of the highest scores noted for each mouse in a specific group up to indicated day of analysis; mean disease duration and mean day of onset were calculated as follows: Mean Disease Duration=Sum of (day of analysis−day of disease onset for each mouse)/(number of mice per group); Mean Day of Onset=(sum of day of disease onset of each mouse)/(number of mice per group). Area under the curve (AUC) of clinical score was calculated using Microsoft Excel and represents disease burden.

Glatiramer Acetate Depot (GA Depot): GA Depot was suspended in water for injection (WFI) and immediately injected intramuscularly (IM) at the indicated dose. Dose of GA Depot are given according to amount of active ingredient (i.e. GA Depot 4 mg contains 4 mg GA).
GA Binding Antibodies Analysis: At day 35 following disease induction, 5 animals from each treatment group were sacrificed. Blood samples were retrieved and serum was isolated and stored at −80° C. (see Tables 3 and 5).

ELISA plates were prepared as following: flat bottom ELISA plates (Nunc) were coated with 100 μl of 50 μg/ml GA in borate buffer (BB) 0.17 M pH 8.0 overnight at 4° C. Wells were emptied and washed with phosphate buffer saline (PBS) containing 0.05% Tween 20 at room temperature. Following the washing 1% BSA was applied for 2 hours at room temperature (RT), for blocking of non-specific binding sites. Subsequently, wells were washed three times with wash solution.

ELISA test was performed as follows: 100 μl of sera samples were diluted 1:1,000 added to wells for 18 hours at 4° C. (sera dilution was performed using PBS containing 1% BSA and 0.05% Tween 20), followed by three washes with phosphate buffer saline (PBS) containing 0.05% Tween 20 at room temperature. Subsequently, 100 μl 1:50,000 diluted alkaline phosphatase conjugated AffinityPure Goat anti-mouse IgG+IgM (H+L) (Jackson Laboratories) was added to the wells and incubated for 2 hours at RT. The wells were washed again three times using wash solution and the color reaction was developed by adding 100 μl of the substrate p-nitrophenyl phosphate (Jackson Laboratories) and incubation for 40-60 min at RT. The reaction was terminated with 30 μl 3N NaOH. The absorbance at 405 nm was then recorded using micro-ELISA reader (Dynatech). Each assay plate contained positive anti-GA serum samples and control of normal mouse serum (N=5).

The results were expressed as Binding Index (BI) according to the following formula: Binding Index=mean optical density of tested serum/mean optical density of control serum. The mean value for normal mice serum was 0.230 OD and the cut off values for Binding Index is 2.0±1.0. Thus, values above 3.0 were considered as positive.

Experimental Design: Studies experimental design is specified in Table 3.

TABLE 3
Experimental design.

| | Test Article, | | | Days of | |
|---|---|---|---|---|---|
| Group | N = 20/Group | Route | Dose | Administration | Solvent |
| 1 | GA Depot | IM | 4 mg | 0, 1* | WFI, 0.2 mL |
| 2 | Copaxone ® | SC | 2 mg | 0-8, 9 in total | N/A |
| 3 | Untreated Control | SC | N/A | 0-8, 9 in total | Saline, 0.1 ml |
| | (saline) | | | | |

*Dose was administered using two injections on consecutive days since there is a maximal volume of injection that can be tolerated in a single injection in mice.

Statistical analysis: Data was analyzed using Microsoft Excel. Each data set was analyzed using single-factor analysis of variance (ANOVA) followed by one tailed student's T-test.

### GA Depot Dose Conversion

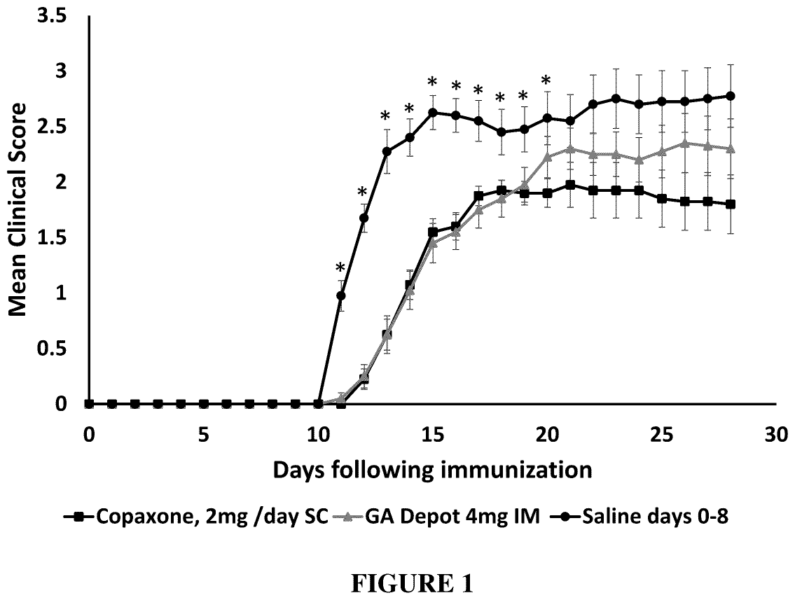

FIG. 1 shows mean clinical score results for saline control (black sphere marker), 2 mg Copaxone® (black square marker) and GA Depot 4 mg (gray triangle marker) groups, as those groups represent the range of the proposed human dose (using an allometric 1:10 scale, as deducted from previous studies) of 0, 20 and 40 mg, respectively. *P<0.05 for all treatment groups compared with untreated control, Single Factor ANOVA followed by one-tailed T Test assuming unequal variance. N=20/group, +/−standard error.

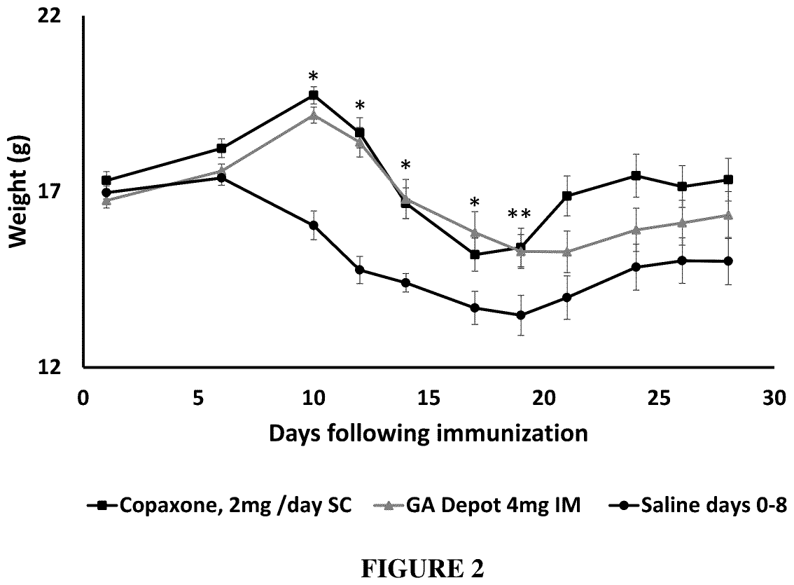

Mean clinical score AUC (area under curve), mean day of onset and mean disease duration were significantly reduced in the GA Depot group and in the Copaxone® group compared with untreated control (Table 4, p<0.05). No statistically significant difference was found between the GA Depot and Copaxone® at any of the computed values (Table 4). At days 11-19, mean clinical score of the saline group was significantly higher than that of all other groups (FIG. 1, p<0.05). At day 20, mean clinical score of the saline group was significantly higher than that of Copaxone® and the GA Depot groups (FIG. 1, p<0.05). Body weight of the GA Depot and Copaxone® treated groups was significantly higher than that of the untreated control at days 10 to 17 following immunization. At day 21, body weight of the Copaxone® group was significantly higher than that of the untreated control group (FIG. 2, P<0.05). *P<0.05 for all treatment groups compared with untreated control. ** P<0.05 for Copaxone® group compared with untreated control. Single Factor ANOVA followed by one-tailed T Test assuming unequal variance. N=20/group, +/−standard error.

TABLE 4
Calculated Values.

| | Maximum | | | | |
|---|---|---|---|---|---|
| | Mean | Mean | Mean | AUC | Survival |
| | Disease | Disease | Day of | Clinical | Rate at |
| Groups | Score | Duration* | Onset* | Score* | Day 28 |
| GA Depot 4 mg IM | 2.55 ± 0.25 | 15.05 ± 0.51 | 12.95 ± 0.51 | 29.85 ± 2.59 | 85% |
| Day 0, 1 | | | | | |
| Copaxone ® | 2.25 ± 0.22 | 15.65 ± 0.23 | 12.35 ± 0.23 | 26.83 ± 2.49 | 90% |
| 2 mg SC, Days 0-8 | | | | | |
| Control (saline, SC) | 3.15 ± 0.24 | 17.80 ± 0.09 | 10.20 ± 0.09 | 42.89 ± 3.22 | 80% |
| D0-D8 | | | | | |

*P < 0.05 for all treatment groups compared with untreated control, Single Factor ANOVA followed by one-tailed T Test assuming unequal variance.
N = 20/group, +/−standard error.

### Immunological Response in Mice Measured by Antibodies to GA

Serum was isolated from mice in MOG-EAE study at day 35 following disease induction. Mice were treated with either the GA Depot (at 4 mg once) or Copaxone® (2 mg/day, days 0-8). Antibodies (Abs) titer was evaluated using an ELISA assay. Results are expressed as Binding Index (BI). N=5. Data as presented in Table 5 demonstrate that mice exposed to Copaxone® or to the GA Depot developed similar titer of total anti-GA antibodies while control mice that were treated with saline had no such antibodies. The antibodies titer was similar in all treatment groups, suggesting a similar immunological response.

TABLE 5
Binding Index.

| | Saline | GA Depot | Copaxone ® |
|---|---|---|---|
| | control | 4 mg | 2 mg |
| Binding Index | 1.71 | 9.48 | 11.14 |

The data presented, comparing the effect of a single administration of the 4 mg GA Depot across to the standard daily administration of 2 mg GA, show similar efficacy between the two dosage regimes. Specifically, the GA Depot showed a clear significant effect of delayed disease onset and amelioration of symptoms, at least as effective as noted for the Copaxone® treated group (see FIG. 1).

In addition, this experiment shows that intramuscular administration of the GA Depot induced a humoral response of anti-GA antibodies, at similar levels as with standard daily subcutaneous injection of GA (see Table 5). Therefore, the similar humoral responses to GA Depot as compared to standard GA injections might represent a similarity in the immunologic response to the GA Depot. This may therefore indicate also equivalent clinical immuno-modulatory therapeutic effects, as can be seen in this EAE study of which the AUC between Copaxone® and GA Depot are not different in statistical significant manner (see Table 4). Therefore, the existence of anti-GA antibodies can serve as a biomarker to the therapeutic bio-availability of the drug, when with a new formulation and administered via a new route.

Overall, data supports that the efficacy of GA Depots at dose of 4 mg GA in MOG-induced EAE is at least comparable to that of Copaxone® and that the immune response to both treatments is similar.

Example 4: Depots of 40 mg or 80 mg GA in Humans Diagnosed with PPMS.
Brief Summary: A prospective, multicenter, single arm, open label, phase IIa study to assess the safety and efficacy of once-a-month long-acting intramuscular injection of 40 mg Glatiramer Acetate (GA Depot) in subjects with Primary Progressive Multiple Sclerosis (PPMS).
Primary endpoints: Safety and tolerability: Assessments of adverse events (AEs); Assessments of injection sites reactions (ISRs).
Secondary endpoints: Efficacy: Time to onset of 12 weeks Confirmed Disease Progression (CDP) assessed by EDSS; Defined as an increase of ≥1 point from baseline EDSS score that was sustained on subsequent visits for at least 12 weeks. MRI assessments: Percent of whole brain volume change; Percent of cortical volume change.
Exploratory endpoints: Efficacy analysis: Change from baseline in Timed 25-foot walk (T25FW). Change from baseline in 9-HPT assessment. MRI assessments: New and enlarging T2 lesions; T2-lesion volume; New and enlarging T1 lesions; T1-lesion volume; Gadolinium (Gd)-lesion number; Gadolinium (Gd)-lesion volume.
Procedures: Subjects visit the site every 4 weeks to receive the GA Depot (40 mg) IM injection administered by a Health care professional (HCP); and are evaluated by the investigator at screening, baseline, 1 week after the second GA Depot injection, 3 months after the first injection and every 3 months thereafter until end of treatment period. A follow up (FU) visit to assess patients' safety is scheduled one month after EoT.
Evaluation criteria: LP: CSF test: Performed at screening visit (IgG OCB) unless the subject has a CSF test prior to screening visit. MRI scans: Performed at screening, week 24 and week 52 (EoT visit). Neurological evaluations, including EDSS, T25FW & 9-HPT performed at screening, baseline, 3 months, and then every 3 months until end of treatment. Adverse events (AEs): monitored throughout the study period. Vital signs: blood pressure (BP) and heart rate recorded at each visit. BP (systolic and diastolic) measured twice at each visit (each measurement separated by a few minutes). Physical examination: A full physical examination performed to ensure suitability per the inclusion and exclusion criteria at screening, baseline, 1 week after the second GA Depot treatment, 3 months after first GA Depot treatment and every 3 months thereafter. Last physical examination performed at FU visit. Clinical laboratory testing: Laboratory tests for chemistry, hematology and urinalysis taken at screening, baseline, 1 month after first treatment, 3, 6, 9 months and at EoT visit. Hematology test include: hemoglobin, red cell count, MCV, hematocrit, MCH, white cell count, differential white cell count, platelet count. Chemistry test include: creatinine, glucose, blood urea nitrogen (BUN), alkaline phosphatase, alanine aminotransferase (ALT/SGPT), aspartate aminotransferase (AST/SGOT), gamma glutamyl transpeptidase (GGT), total bilirubin, protein, albumin, albumin/globulin ratio, sodium, potassium, chloride, calcium, uric acid, CPK, cholesterol, triglycerides. Urinalysis-stick test performed at site.

Each patient is compared to his individual baseline test taken at screening visit. When in the subject's best interest, additional tests may be performed at the discretion of the Investigator to ensure their good health.

Antibodies testing: Blood samples for total Glatiramer Acetate IgG binding antibodies and detection of neutralizing antibodies to Glatiramer Acetate taken at baseline, 1 month after the first GA Depot injection, 3 months, and every 3 months thereafter. 12-lead ECG performed at screening. The ECG is recorded while the subject is resting in supine position. 6 limb leads, as specified by Einthoven (I, II and III) and Goldberger (aVR, aVL, aVF), and 6 pre-cordial leads (V1-V6), according to Wilson, are used. The investigator assesses the parameters HR, RR, PQ, QRS, QT and QTc. Additionally, the occurrence of de- or repolarization disorders, arrhythmic disorders or other abnormalities is assessed and recorded. Chest x-ray performed at screening if not performed within 6 months prior to screening visit.

Treatment location and patient supervision: Study injections are administered and monitored at site. Subjects stay at the study site for observation for 1 hour following the first GA Depot injection and half an hour following the subsequent GA Depot injections to ensure their well-being.

Trial treatments, dosage, and dosage regimen of the investigational products: All enrolled subjects are treated with GA Depot (40 mg) IM at 4 week intervals for a total of 52 weeks of treatment.
Expected duration of subject participation, sequence, and duration of all trial periods including follow-up: The study duration for an individual subject is up to 14 months, consisting of the screening period and 12 months of treatment followed by a follow up visit as follows: up to 4 weeks of screening period (weeks-4 to 0), 52 weeks of open-label treatment period, an EoT visit at week 52 and a follow-up visit 4 weeks after the EoT visit.
Subject inclusion criteria: Subjects must fulfill all the following criteria before being included in the study: Male or female subjects diagnosed with PPMS. Diagnosis of PPMS consistent with the McDonald Criteria (revisions of 2010); Age between 18 and 60 years (inclusive); Subjects diagnosed with PPMS for at least 1 year and a sustained increment of ≥1 point in the EDSS score in the last year prior to screening; EDSS≥2 and ≤5.5 (Pyramidal or Cerebellar FS≥2); Documented history or the presence at screening of >1 oligoclonal band (OCB) if quantitative testing was done, or OCB+ if no quantitative testing was done, and/or positive IgG index in the cerebrospinal fluid (CSF); Subjects with at least 1 gadolinium-enhancing lesion on baseline MRI and/or documented in previous MRI within 12 months prior to screening visit; Women of child bearing potential must have a negative urine pregnancy test at screening and use an adequate contraceptive method throughout the study; Ability to provide written informed consent.
Subject exclusion criteria: Subjects are to be excluded from the study if they display any of the following criteria: RRMS, SPMS, or PRMS; Documented history of relapse events; Any relevant medical, surgical, or psychiatric condition, laboratory value, or concomitant medication which, in the opinion of the investigator, makes the subject unsuitable for study entry or potentially unable to complete all aspects of the study; Contraindications or inability to successfully undergo magnetic resonance imaging (MRI) scanning; Subjects diagnosed with any systemic autoimmune disease other than MS that may impact the CNS with MS like lesions such as Sarcoidosis, Sjögren's syndrome, Systemic Lupus Erythematosus (SLE), Lyme disease, APLA syndrome, etc. Subjects with stable local/organ autoimmune disease such as psoriasis, Cutaneous Lupus erythematosus, thyroiditis (Hashimoto, grave) etc. may be considered eligible upon the PI's discretion; Severe anemia (hemoglobin<10 g/dL); Abnormal renal function (serum creatinine >1.5×ULN or creatinine clearance <30 ml/min); Abnormal liver function (transaminases >2×ULN); Pregnant or breast-feeding women; Treatment with any kind of steroids during the last month prior to screening visit; History of any anaphylactic reaction and/or serious allergic reaction following a vaccination, a known hypersensitivity to any component of the study drug, e.g. glatiramer acetate (GA), polylactic-co-glycolic acid (PLGA), polyvinyl alcohol (PVA); Known or suspected history of drug or alcohol abuse; Known as positive for HIV, hepatitis, VDRL, or tuberculosis; Active malignant disease of any kind. However, a patient, who had a malignant disease in the past, was treated and is currently disease-free for at least 7 years, may be considered eligible, upon the PI and sponsor discretion; Previous treatment with B-cell-targeting therapies (e.g. rituximab, ocrelizumab, atacicept, belimumab or ofatumumab) 6 months prior to screening; Previous treatment with cladribine within 2 years prior to screening visit; Previous treatment with azathioprine, mitoxantrone or methotrexate within 6 months prior to screening visit; Previous treatment with lymphocyte-trafficking modifiers (e.g. natalizumab, fingolimod) within 6 months prior to screening visit. Subjects should have a total lymphocyte count within normal range; Previous treatment with beta interferons, intravenous immunoglobulin, plasmapheresis within 2 months prior to screening visit; Previous treatment with any glatiramer acetate therapy within 3 months prior to screening visit; Uncontrolled diabetes; Participation in an investigational study drug within 30 days prior to study entry.
Formulation and dosing of study drug: GA Depot is a combination of extended-release microspheres containing GA for injection and diluent for parenteral use. The extended-release microsphere formulation is a white to off-white, free-flowing powder in dosage strength of 40 mg GA per vial. GA is micro-encapsulated in poly(D,L-lactide-co-glycolide) (50:50) acid terminated, at a concentration of 80 mg GA per gram of microspheres plus 10% overage to compensate for withdrawal losses. The diluent for parenteral use is water for injection. The microspheres are suspended in the diluent prior to injection.
Test treatment: IP: long-acting Glatiramer Acetate (GA Depot). Formulation: 550 mg of lyophilized PLGA-encapsulated glatiramer acetate containing 40 mg GA. Water for injection (WFI): 10 ml ampule. 1.6 ml WFI are used for suspension of one 40 mg GA Depot vial to reach a final 2 ml volume of suspension for injection. Route of administration: Intramuscular. Unit dose: 40 or 80 mg GA Depot. Dosage schedule: Each subject receives 40 or 80 mg of GA Depot administered IM once every 4 weeks, 13 times for a total of 52 weeks of treatment.
Specification of efficacy/pharmacology parameters: The following clinical and efficacy parameters are assessed: EDSS score; MRI assessments (whole brain volume, cortical brain volume, new and enlarging T2 lesions, T2-lesion volume, new and enlarging T1 lesions, T1-lesion volume, Gadolinium (Gd)-lesion number, Gadolinium (Gd)-lesion volume); Timed 25-foot walk (T25FW); and/or 9-HPT.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1. A method of treating a patient diagnosed with primary progressive multiple sclerosis (PPMS) or secondary progressive multiple sclerosis (SPMS), the method comprising the step of administering or implanting a sustained release depot formulation comprising a therapeutically effective amount of a pharmaceutically acceptable salt of glatiramer to the patient.

2. The method of claim 1, wherein the patient:
(i) has been diagnosed with PPMS for at least 1 year and a sustained increment of ≥1 point in the EDSS score in the last year;
(ii) has an EDSS score between 2 and 5.5, inclusive (Pyramidal or Cerebellar FS≥2);
(iii) has a documented history of, or the presence of more than 1 oligoclonal band (OCB) if quantitative testing was done, or OCB+ if no quantitative testing was done, and/or positive IgG index in the cerebrospinal fluid (CSF);
(iv) has at least 1 gadolinium-enhancing lesion on MRI and/or at least 1 gadolinium-enhancing lesion documented within the previous year on MRI; or
(v) any combination of (i) to (iv).

3. The method of claim 1, wherein treating a patient diagnosed with PPMS or SPMS comprises suppressing or alleviating at least one symptom selected from the group consisting of impaired walking capability, weakness of the leg, stiffness of the leg, impaired balance, impaired coordination, impaired memory, impaired cognitive function, a difficulty to swallow, impaired vision, general fatigue, pain, impaired bladder function, impaired bowel function, and any combination thereof.

4. The method of claim 1, wherein treating a patient diagnosed with PPMS or SPMS comprises:
(i) increasing the time to onset of 12 week Confirmed Disease Progression (CDP) of the patient assessed by Expanded Disability Status Scale (EDSS), compared to baseline or untreated control;
(ii) decreasing whole brain volume change or cortical volume change of the patient, compared to baseline or untreated control;
(iii) decreasing the time needed for the patient to complete a timed 25-foot walk (T25FW) test, compared to baseline or untreated control;
(iv) decreasing the time needed for the patient to complete a 9-Hole Peg Test (9-HPT), compared to baseline or untreated control;
(v) decreasing the number of new or enlarging T2 lesions in the brain of the patient, compared to baseline or untreated control;
(vi) decreasing the volume of T2 lesions in the brain of the patient, compared to baseline or untreated control;
(vii) decreasing the number of new or enlarging T1 lesions in the brain of the patient, compared to baseline or untreated control;
(viii) decreasing the volume of T1 lesions in the brain of the patient, compared to baseline or untreated control;
(ix) decreasing the number or volume of Gadolinium (Gd) lesions in the brain of the patient, compared to baseline or untreated control;
(x) reducing the rate of progression of PPMS or SPMS, compared to baseline or untreated control;
(xi) preventing further progression of PPMS or SPMS, compared to baseline or untreated control; or
(xii) any combination of (i) to (xi).

5. The method of claim 4, wherein the patient has an EDSS score of <5.5 and CDP is at least a 1 point increase of the EDSS score.

6. The method of claim 4, wherein the patient has an EDSS score of 5.5-10 and CDP is at least a 0.5 point increase of the EDSS score.

7. The method of claim 4, wherein the baseline is a period of 12 weeks or more prior to treatment by the depot formulation.

8. The method of claim 1, wherein the depot formulation is administered or implanted intramuscularly.

9. The method of claim 1, wherein the depot formulation comprises 40 mg to 80 mg dose of the pharmaceutically acceptable salt of glatiramer.

10. The method of claim 9, wherein the depot formulation comprises a 40 mg dose of the pharmaceutically acceptable salt of glatiramer.

11. The method of claim 1, wherein the depot formulation is administered in a therapeutically effective regime of once every 1 to 15 weeks.

12. The method of claim 11, wherein the depot formulation is administered once every 4 weeks.

13. The method of claim 11, wherein the depot formulation is repeatedly administered during 1 year or more.

14. The method of claim 1, wherein the pharmaceutically acceptable salt of glatiramer is glatiramer acetate (GA).

15. The method of claim 1, wherein the depot formulation comprises 20% to 30% solids.

16. The method of claim 1, wherein the depot formulation comprises a Poly(Lactide-co-Glycolide) (PLGA) copolymer.

17. The method of claim 16, wherein the depot formulation comprises 550 mg PLGA copolymer per 40 mg of the pharmaceutically acceptable salt of glatiramer.

18. The method of claim 1, wherein the depot formulation, in phosphate buffered saline (PBS, pH 7.4), in a closed vial, at 37° C., while stirring:
(i) releases about 15% to about 30% of the glatiramer salt within 1 week;
(ii) releases about 30% to about 50% of the glatiramer salt within 2 weeks;
(iii) releases about 50% to about 90% of the glatiramer salt within 3 weeks;
(iv) releases about 90% to about 100% of the glatiramer salt within 4 weeks; or
(v) any combination of (i) to (iv).

19. A method of increasing the tolerability or convenience of glatiramer treatment, or increasing the adherence to glatiramer treatment, in a patient diagnosed with progressive multiple sclerosis (PPMS) or secondary progressive multiple sclerosis (SPMS) and receiving an immediate-release or bolus glatiramer formulation, the method comprising reducing the frequency of glatiramer administration by administering a therapeutically effective depot formulation of a pharmaceutically acceptable salt of glatiramer to the patient, so as to thereby increase the tolerability, convenience or adherence of the patient to glatiramer treatment.

20. A method of preparing a sustained release depot formulation comprising an internal aqueous phase comprising a therapeutically effective amount of a pharmaceutically acceptable salt of glatiramer, a water immiscible polymeric phase comprising a biodegradable or non-biodegradable polymer and an external aqueous phase for treating a patient diagnosed with primary progressive multiple sclerosis (PPMS) or secondary progressive multiple sclerosis (SPMS), the method comprises a water-in oil-in water double emulsification process.
